SEQUENCE LISTING (6 sequences)

SEQ ID NO: 1 - DNA, 21 bp, Bacteroides thetaiotaomicron
atagcctttcgaaagraagat

SEQ ID NO: 2 - DNA, 21 bp, Bacteroides thetaiotaomicron
ccagtatcaactgcaatttta

SEQ ID NO: 3 - DNA, 19 bp, Methanobrevibacter smithii
tgagatgtccggcgttgaa

SEQ ID NO: 4 - DNA, 20 bp, Methanobrevibacter smithii
aagccatgcaagtcgaacga

SEQ ID NO: 5 - DNA, 26 bp, Desulfovibrio piger
ctagggtgttctaatcatcatcctac

SEQ ID NO: 6 - DNA, 21 bp, Desulfovibrio piger
ttgagtttcagccttgcgacc

(19) United States
(12) Patent Application Publication
(10) Pub. No.: US 2010/0048595 A1
(43) Pub. Date: Feb. 25, 2010

(54) USE OF ARCHAEA TO MODULATE THE NUTRIENT HARVESTING FUNCTIONS OF THE GASTROINTESTINAL MICROBIOTA

(75) Inventors: Jeffrey I. Gordon, St. Louis, MO (US); Sparrow Buck Samuel, St. Louis, MO (US)

Correspondence Address: POLSINELLI SHUGHART PC, 100 SOUTH FOURTH STREET, SUITE 100, SAINT LOUIS, MO, 63102-1825, US

(73) Assignee: WASHINGTON UNIVERSITY IN ST. LOUIS, St. Louis, MO (US)

(21) Appl. No.: 11/909,126

(22) PCT Filed: Mar. 22, 2006
(86) PCT No.: PCT/US06/10289

§ 371 Date: Sep. 28, 2009

Related U.S. Application Data
(60) Provisional application No. 60/664,558, filed on Mar. 23, 2005.

(51) Int. Cl. A61K 31/505; A61K 31/4164; A61K 31/255; A61K 31/195; A61K 31/35; A61K 31/40; A61K 31/404; A61P 3/04

(52) U.S. Cl. 514/275; 514/399; 514/517; 514/567; 514/460; 514/423; 514/415

(57) ABSTRACT

The invention generally relates to the use of archaea to modulate nutrient harvesting in a subject. In particular, the invention provides methods that use archaea to modulate the nutrient harvesting functions of the microbiota in the subject's gastrointestinal tract.

### GOVERNMENTAL RIGHTS

This invention was made with Government support under Contracts No. DK70977 and DK30292 awarded by the National Institutes of Health. The Government has certain rights in this invention.

### FIELD OF THE INVENTION

The current invention generally relates to the use of mesophilic methanogenic archaea to modulate nutrient harvesting in a subject. In particular, the invention provides methods that use archaea to modulate the nutrient harvesting functions of the microbiota in the subject's gastrointestinal tract.

### BACKGROUND OF THE INVENTION

### I. Obesity Problem and Current Approaches

According to the Center for Disease Control (CDC), over sixty percent of the United States population is overweight, and almost twenty percent are obese. This translates into 38.8 million adults in the United States with a Body Mass Index (BMI) of 30 or above. Obesity is also a world-wide health problem with an estimated 500 million overweight adult humans [body mass index (BMI) of 25.0-29.9 kg/m²] and 250 million obese adults (1). This epidemic of obesity is leading to worldwide increases in the prevalence of obesity-related disorders, such as diabetes, hypertension, as well as cardiac pathology, and non-alcoholic fatty liver disease (NAFLD; 2-4).

According to the National Institute of Diabetes, Digestive and Kidney Diseases (NIDDK) approximately 280,000 deaths annually are directly related to obesity. The NIDDK further estimated that the direct cost of healthcare in the U.S. associated with obesity is $51 billion. In addition, Americans spend $33 billion per year on weight loss products. In spite of this economic cost and consumer commitment, the prevalence of obesity continues to rise at alarming rates. From 1991 to 2000, obesity in the U.S. grew by 61%.

Although the physiologic mechanisms that support development of obesity are complex, the medical consensus is that the root cause relates to an excess intake of calories compared to caloric expenditure. While the treatment seems quite intuitive, dieting is not an adequate long-term solution for most people; about 90 to 95 percent of persons who lose weight subsequently regain it. Although surgical intervention has had some measured success, the various types of surgeries have relatively high rates of morbidity and mortality.

Pharmacotherapeutic principles are limited. In addition, because of undesirable side effects, the FDA has had to recall several obesity drugs from the market. Those that are approved also have side effects. Currently, two FDA-approved anti-obesity drugs are orlistat, a lipase inhibitor, and sibutramine, a serotonin reuptake inhibitor. Orlistat acts by blocking the absorption of fat into the body. An unpleasant side effect with orlistat, however, is the passage of undigested oily fat from the body. Sibutramine is an appetite suppressant that acts by altering brain levels of serotonin. In the process, it also causes elevation of blood pressure and an increase in heart rate. Other appetite suppressants, such as amphetamine derivatives, are highly addictive and have the potential for abuse. Moreover, different subjects respond differently and unpredictably to weight-loss medications.

In summary, current surgical and pharmacotherapy treatments are problematic. Novel non-cognitive strategies are needed to prevent and treat obesity and obesity-related disorders. Toward that end, modulation of gastrointestinal microbial populations represents a non-cognitive strategy for influencing energy storage and metabolism in a subject whose potential has not fully been characterized.

### II. Gastrointestinal Microbiota

Humans are host to a diverse and dynamic population of microbial symbionts, with the majority residing within the distal intestine. The gut microbiota contains representatives from nine known divisions of the domain Bacteria, with an estimated 800 bacterial species present; it is dominated by members of two divisions of the domain Bacteria, the Bacteroidetes and the Firmicutes. The density of colonization increases by eight orders of magnitude from the proximal small intestine (10³) to the colon (10¹¹). The distal intestine is an anoxic bioreactor whose microbial constituents help the host by providing a number of key functions: e.g., breakdown of otherwise indigestible plant polysaccharides and regulating host storage of the extracted energy (5, 6); biotransformation of conjugated bile acids (7) and xenobiotics; degradation of dietary oxalates (8); synthesis of essential vitamins (9); and education of the immune system (10).

Dietary fiber is a key source of nutrients for the microbiota. Monosaccharides are absorbed in the proximal intestine, leaving dietary fiber that has escaped digestion (e.g. resistant starches, fructans, cellulose, hemicelluloses, pectins) as the primary carbon sources for microbial members of the distal gut. Fermentation of these polysaccharides yields short-chain fatty acids (SCFAs; mainly acetate, butyrate and propionate) and gases (H₂ and CO₂). These end products benefit humans (11). For example, SCFAs are an important source of energy, as they are readily absorbed from the gut lumen and are subsequently metabolized in the colonic mucosa, liver, and a variety of peripheral tissues (e.g., muscle) (11). SCFAs also stimulate colonic blood flow and the uptake of electrolytes and water (11).

### III. Methanogens

Methanogens are members of the domain Archaea (FIG. 1) (12). Methanogens thrive in many anaerobic environments together with fermentative bacteria. These habitats include natural wetlands as well as man-made environments, such as sewage digesters, landfills, and bioreactors. Hydrogen-consuming, mesophilic methanogens are also present in the intestinal tracts of many invertebrate and vertebrate species, including termites, birds, cows, and humans (13-16). Using methane breath tests, clinical studies estimate that between 50 and 80 percent of humans harbor methanogens (17-19).

Culture- and non-culture-based enumeration studies have demonstrated that members of the Methanobrevibacter genus are prominent gut mesophilic methanogens (14). The most comprehensive enumeration of the adult human colonic microbiota reported to date (20) found a single predominant archaeal species, Methanobrevibacter smithii. This gram-positive-staining Euryarchaeote can comprise up to 10¹⁰ cells/g feces in healthy humans, or ˜10% of all anaerobes in the colons of healthy adults (21-24). Methanosphaera stadtmanae and Sulfolobus group crenarchaeotes can also be minor archaeal members of the microbiota (23-25).

A focused set of nutrients are consumed for energy by methanogens: primarily H₂/CO₂, formate, acetate, but also methanol, methylated sulfur compounds, methylated amines and pyruvate (26, 27). These compounds are typically converted to CO₂ and methane (e.g. acetate) or reduced with H₂ to methane alone (e.g. methanol or CO₂). Some methanogens are restricted to utilizing only H₂/CO₂ (e.g. Methanobrevibacter arbophilicus), or methanol (e.g. M. stadtmanae). Other more ubiquitous methanogens exhibit greater metabolic diversity, like Methanosarcina species (28, 29). In vitro studies suggest that M. smithii is intermediate in this metabolic spectrum, consuming H₂/CO₂ and formate as energy sources (23, 24, 30).

### IV. Anaerobic Microbial Fermentation in the Mammalian Intestine

Fermentation of dietary fiber is accomplished by syntrophic interactions between microbes linked in a metabolic food web, and is a major energy-producing pathway for members of the Bacteroidetes and the Firmicutes. Bacteroides thetaiotaomicron has previously been used as a model bacterial symbiont for a variety of reasons: (i) it effectively ferments a range of otherwise indigestible plant polysaccharides in the human colon (31); (ii) it is genetically manipulatable (32); and, (iii) it is a predominant member of the human distal intestinal microbiota (20, 33). Its 6.26 Mb genome has been sequenced (34): the results reveal that B. thetaiotaomicron has the largest collection of known or predicted glycoside hydrolases of any prokaryote sequenced to date (226 in total; by comparison, our human genome only encodes 98 known or predicted glycoside hydrolases). B. thetaiotaomicron also has a significant expansion of outer membrane polysaccharide binding and importing proteins (163 paralogs of two starch binding proteins known as SusC and SusD), as well as a large repertoire of environmental sensing proteins [e.g. 50 extra-cytoplasmic function (ECF)-type sigma factors; 25 anti-sigma factors, and 32 novel hybrid two-component systems; (34)]. Functional genomics studies of B. thetaiotaomicron in vitro and in the ceca of gnotobiotic mice, indicates that it is capable of very flexible foraging for dietary (and host) polysaccharides, allowing this organism to have a broad niche and contributing to the functional stability of the microbiota in the face of changes in the diet (35).

In vitro biochemical studies of B. thetaiotaomicron and closely related Bacteroides species (B. fragilis and B. succinogenes) indicate that their major end products of fermentation are acetate, succinate, H₂ and CO₂ (36-38). Small amounts of pyruvate, formate, lactate and propionate are also formed (FIG. 2).

### V. Removal of Hydrogen from the Intestinal Ecosystem is Important for Efficient Microbial Fermentation

Anaerobic fermentation of sugars causes flux through glycolytic pathways, leading to accumulation of NADH (via glyceraldehyde-3P dehydrogenase) and the reduced form of ferredoxin (via pyruvate:ferredoxin oxidoreductase). B. thetaiotaomicron is able to couple NAD⁺ recovery to reduction of pyruvate to succinate (via malate dehydrogenase and fumarase reductase), or lactate (via lactate dehydrogenase) (FIG. 2; (36-38)). Oxidation of reduced ferredoxin is easily coupled to production of H₂. However, H₂ formation is, in principle, not energetically feasible at high partial pressures of the gas (39). In other words, lower partial pressures of H₂ (1-10 Pa) allow for more complete oxidation of carbohydrate substrates (40). The host removes some hydrogen from the colon by excretion of the gas in the breath and as flatus. However, the primary mechanism for eliminating hydrogen is by interspecies transfer from bacteria by hydrogenotrophic methanogens (40, 41). Formate and acetate can also be transferred between some species, but their transfer is complicated by their limited diffusion across the lipophilic membranes of the producer and consumer (42). In areas of high microbial density or aggregation like in the gut, interspecies transfer of hydrogen, formate and acetate is likely to increase with decreasing physical distance between microbes (40).

Methanogen-mediated removal of hydrogen can have a profound impact on bacterial metabolism. Not only does re-oxidation of NADH occur, but end products of fermentation undergo a shift from a mixture of acetate, formate, H₂, CO₂, succinate and other organic acids to predominantly acetate and methane with small amounts of succinate (40). This facilitates disposal of reducing equivalents, and produces a potential gain in ATP production due to increased acetate levels. For example, a reduction in hydrogen allows Clostridium butyricum to acquire 0.7 more ATP equivalents from fermentation of hexose sugars (39). Co-culture of M. smithii with a prominent cellulolytic ruminal bacterial species, Fibrobacter succinogenes S85, results in augmented fermentation, as manifested by increases in the rate of ATP production and organic acid concentrations (43). Co-culture of M. smithii association with Ruminococcus albus eliminates NADH-dependent ethanol production from acetyl-CoA, thereby skewing bacterial metabolism towards production of acetate, which is more energy yielding (44). H₂-producing fibrolytic bacterial strains from the human colon exhibit distinct cellulose degradation phenotypes when co-cultured with M. smithii, indicating that some bacteria are more responsive to syntrophy with methanogens (45).

While there is suggestive evidence that methanogens cooperate metabolically with members of Bacteroides, no in vivo studies have elucidated the impact of this relationship on a host's energy storage or on the specificity and efficiency of carbohydrate metabolism. For example, one study noted that co-culture of M. smithii with a B. thetaiotaomicron strain led to increased degradation of broad bean cell walls (46). But there are no reports of comparable studies in vivo, or of assays of the reciprocal impact of any methanogen and a saccharolytic bacterium on each another's transcriptomes and metabolomes within their intestinal habitat.

### SUMMARY OF THE INVENTION

Briefly, the present discovery was made by studying the syntrophic relationships between the gastrointestinal archaea and the gastrointestinal bacteria. By studying this relationship, the applicants have discovered that the archaea modulate the polysaccharide degrading properties of the microbiota. In particular, the applicants have discovered that the archaea change prioritized bacterial utilization of polysaccharides commonly encountered in our modern diets by altering the transcriptome and the metabolome of a predominant bacterial component of the host's gastrointestinal microbiota. In addition, the applicants also discovered a link between this archaeon and enhanced host recovery and storage of energy from the diet.

Among the several aspects of the current invention, therefore, is the provision of methods that may be utilized to regulate the efficiency and specificity of carbohydrate metabolism in a subject. In certain aspects of the invention, a method for promoting weight loss in a subject is provided. The method typically comprises altering the archaeal population in the subject's gastrointestinal tract such that microbial-mediated carbohydrate metabolism or the efficiency of microbial-mediated carbohydrate metabolism is decreased in the subject, whereby decreasing microbial-mediated carbohydrate metabolism or the efficiency of microbial-mediated carbohydrate metabolism promotes weight loss in the subject. In other aspects of the invention, a method is provided for altering the specificity or efficiency of microbial-mediated carbohydrate metabolism by increasing or decreasing the archaeal population in the subject's gastrointestinal tract.

Yet another aspect of the invention provides methods that may be used to treat diseases or disorders. In certain aspects of the invention, a method for treating obesity or an obesity related disorder is provided. The method typically comprises altering the archaeal population in the subject's gastrointestinal tract such that microbial-mediated carbohydrate metabolism is decreased in the subject, whereby decreasing microbial-mediated carbohydrate metabolism promotes weight loss in the subject. Another aspect of the invention provides use of the amount of archaea in the gut as a biomarker for use in predicting whether a subject is at risk for becoming obese or suffering from an obesity-related condition. In other aspects of the invention, a method for reducing the symptoms of irritable bowel syndrome arising from an inability to ferment dietary polysaccharides is provided. The method typically comprises altering the archaeal population in the subject's gastrointestinal tract. In other aspects of the invention, a general method for altering the representation of bacterial components of the host microbiota is provided.

Other aspects and embodiments of the invention are described in more detail herein.

### BRIEF DESCRIPTION OF THE DRAWINGS

FIG. 1 depicts a schematic illustrating a phylogenetic tree based on 16S ribosomal RNA sequences. Few archaeal genomes have been sequenced (21 vs. 201 in Bacteria, as of March 2005; number of sequenced genomes in division indicated in parentheses). Animal-associated Archaea cluster primarily within the Methanobacterium division, which has only one sequenced member, the M. stadtmanae genome (56).

FIG. 2 depicts a schematic of B. thetaiotaomicron fermentation pathways and production of substrates for methanogens. The major end products of B. thetaiotaomicron fermentation are acetate, succinate and hydrogen (H₂), though propionate and formate are also produced at lower levels. Degradation of dietary fiber through glycolytic pathways increases levels of NADH that cannot be oxidized to NAD⁺ when excess hydrogen is present. Methanogens can consume H₂/CO₂, formate, and acetate via interspecies metabolite transfer, which may promote fermentation in the distal gut. The key enzymes involved in this process include: 1) pyruvate:ferridoxin oxidoreductase; 2) phosphotransacetylase and acetate kinase; 3) phosphobutyryltransferase and butyrate kinase; 4) pyruvate:formate lyase; 5) lactate dehydrogenase; 6) malate dehydrogenase and succinate dehydrogenase; and 7) succinyl-CoA synthetase and propionyl-CoA decarboxylase.

FIG. 3 depicts a graph illustrating that co-colonization with Methanobrevibacter smithii and Bacteroides thetaiotaomicron enhances the representation of both species in the distal intestines of germ-free (GF) mice. The density of colonization was defined using quantitative PCR of DNA isolated from the cecal and colonic contents of mice colonized with one or the other or both species (‘mono- and bi-associated’ animals; n=5/group/experiment; three independent experiments; each cecal sample assayed in triplicate; mean values±SEM plotted; *, p<0.05 vs. mono-associated controls). Abbreviations: Bt, B. thetaiotaomicron; Ms, M. smithii.

FIG. 4 depicts a graph showing the Clusters of Orthologous Groups (COGs) categorization of B. thetaiotaomicron genes up- or down-regulated in the ceca of GF mice in the presence of M. smithii. All genes designated by GeneChip analysis as being significantly (p<0.05) up- or down-regulated in B. thetaiotaomicron/M. smithii mice compared to B. thetaiotaomicron mono-associated mice have been placed into COGs.

FIG. 5 illustrates that M. smithii focuses B. thetaiotaomicron foraging of polyfructose-containing glycans in the distal gut. Panel A presents GeneChip analysis of RNA isolated from cecal contents of individual mice colonized with B. thetaiotaomicron±M. smithii (n=5/group). Shown is unsupervised hierarchical clustering (dChip) of the 57 B. thetaiotaomicron glycoside hydrolases (GH) and polysaccharide lysases (PL) downregulated in the presence of M. smithii. Each column in each group represents data obtained from a cecal sample harvested from an individual mouse, while each row represents a B. thetaiotaomicron (Bt) gene. Panel B presents a schematic of the B. thetaiotaomicron polyfructose degradation gene cluster induced in the presence of M. smithii. Gene ID numbers are presented below the arrows representing the genes. Panel C presents a graph illustrating the biochemical analysis of fructan and glucan levels in cecal contents (n=5 mice/group; each cecal sample assayed in duplicate; mean values±SEM plotted; *, p<0.05).

FIG. 6 illustrates the effect of co-colonization with the sulfate-reducing, H₂-consuming, human gut-associated bacterium Desulfobacter piger on the B. thetaiotaomicron transcriptome. Panel A depicts a graph showing the fold differences in the expression of selected B. thetaiotaomicron genes in the ceca of B. thetaiotaomicron/M. smithii or B. thetaiotaomicron/D. piger bi-associated mice versus B. thetaiotaomicron mono-associated animals as determined by qRT-PCR. Mean values±SEM are plotted; *, p<0.05 vs. B. thetaiotaomicron. Panel B shows GeneChip analysis of B. thetaiotaomicron glycoside hydrolase genes whose expression was significantly different (p<0.05) in the presence of D. piger compared to mono-associated controls. Fold-difference was defined by GeneChip analysis. Each column in each group represents data obtained from a cecal sample harvested from an individual mouse. Abbreviations: Bt, B. thetaiotaomicron; Ms, M. smithii; Dp, D. piger.

FIG. 7 depicts a graph illustrating the effects of M. smithii on glycan foraging by B. thetaiotaomicron. Shown is gas chromatography-mass spectrometry (GC-MS) analysis of neutral and amino sugars present in the cecal contents of germ free, B. thetaiotaomicron/M. smithii bi-associated, and mono-associated mice (n=4/group). Mean values±SEM are plotted; *, p<0.05.

FIG. 8 illustrates that bi-association with B. thetaiotaomicron and M. smithii increases B. thetaiotaomicron production of acetate and formate. Panel A presents a schematic of the short chain fatty acid (SCFA) production pathway. Boxed numbers present the qRT-PCR fold change of M. smithii on the expression of selected B. thetaiotaomicron genes encoding enzymes involved in fermentation of polyfructose-containing glycans: fructofuranosidases, BT1765/BT1759; fructokinase, BT1757; phosphofructokinase, BT0307; pyruvate:formate lyase, BT4738; acetate kinase, BT3963, methylmalonyl-CoA decarboxylase, BT1688; butyrate kinase, BT2552. Enzyme classification (E.C.) numbers are provided in parentheses. Dotted lines indicate multi-step pathways. [B. thetaiotaomicron transcription of fructofuranosidases, acetate kinase, puruvate:formate lyase and butyrate kinase remains constant if the colonization period is extended from 14d to 28d.] Panel B shows a graph of the levels of cecal SCFAs in the mono- and bi-associated mice (n=5/group; each sample assayed by GC-MS in duplicate; mean values±SEM plotted). Panel C depicts a graph of the qRT-PCR analysis of the in vivo expression of M. smithii genes in a cluster (lower panel) containing formate transporter/dehydrogenase (fdhCAB) and tungsten-containing formylmethanofuran dehydrogenase subunits (fwdEFDBAC) (n=5/group; each sample assayed in triplicate; mean values±SEM plotted; *, p<0.05).

FIG. 9 depicts a graph showing the preferential consumption of formate by M. smithii during in vitro culture. Growth of M. smithii in chemostats containing complex methanogen medium (MBC) supplemented with formate and acetate under a constant stream of H₂/CO₂ gas (4:1). Aliquots were taken periodically to measure optical density (OD₆₀₀) and levels of organic acids (ppm, parts per million, assayed by ionization chromatography).

FIG. 10 presents graphs illustrating that co-colonization of mice with M. smithii and B. thetaiotaomicron enhances host energy storage. Panel A presents GC-MS analyses of acetate in sera obtained by retro-orbital phlebotomy from fasted (4h) 12-week-old male B. thetaiotaomicron mono-associated, and bi-associated [B. thetaiotaomicron/M. smithii or B. thetaiotaomicron/D. piger (Dp)] GF mice (n=5/group/experiment; two independent experiments; mean values±SEM are plotted). Panel B presents liver triglyceride levels (n=5/group; each assayed in duplicate; mean values±SEM plotted). Panel C presents epididymal fat pad weights (n=5/group/experiment; two independent experiments; mean values±SEM plotted; *, p<0.05; **, p<0.01; ***, p<0.005).

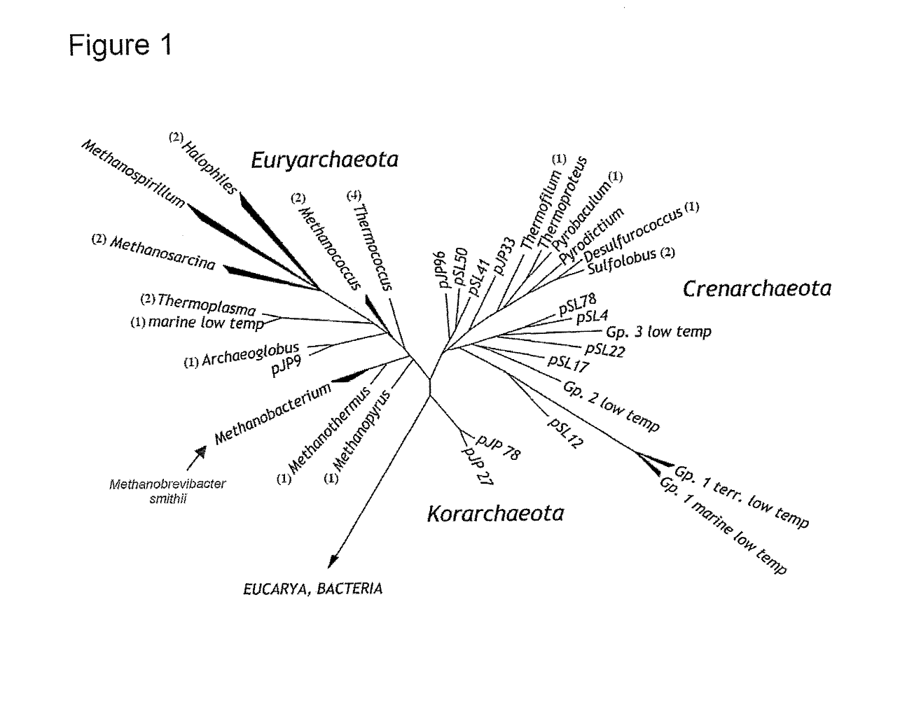

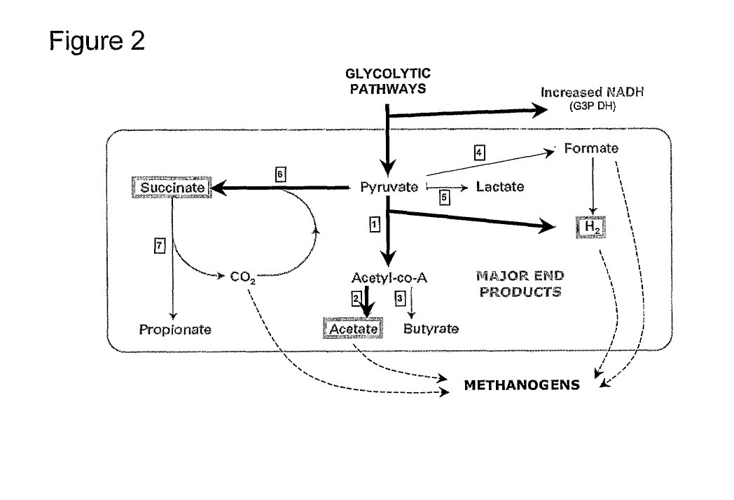

### DETAILED DESCRIPTION OF THE PREFERRED EMBODIMENTS

The applicants have discovered that the archaea modulate the polysaccharide degrading properties of the microbiota, enhancing harvest and storage of dietary calories by the host. In particular, the applicants have discovered that the archaea improve the metabolism of otherwise indigestible dietary polysaccharides by altering the transcriptome and the metabolome of a predominant bacterial component of the host's gastrointestinal microbiota. Taking advantage of these discoveries, the present invention provides compositions and methods that may be employed for modulating carbohydrate metabolism or the efficiency of carbohydrate metabolism in a subject. Advantageously, because carbohydrate metabolism and its efficiency can be regulated by the methods of the invention, the invention also provides methods for promoting weight loss or disease management in a subject.

### (A) Alteration of the Gastrointestinal Archaeon Population

One aspect of the present invention provides a method for decreasing microbial-mediated carbohydrate metabolism or for decreasing the efficiency of microbial-mediated carbohydrate metabolism in a subject by altering the archaeon population in the subject's gastrointestinal tract. Because carbohydrate metabolism or the efficiency of carbohydrate metabolism may be decreased, the invention also provides methods for promoting weight loss in the subject. To promote weight loss in a subject, the archaeon population is altered such that microbial-mediated carbohydrate metabolism or its efficiency is decreased in the subject, whereby decreasing microbial-mediated carbohydrate metabolism or its efficiency promotes weight loss in the subject.

Accordingly, in one embodiment, the subject's gastrointestinal archaeon population is altered so as to promote weight loss in the subject. Typically, the presence of at least one genera of archaeon that resides in the gastrointestinal tract of the subject is decreased. In most embodiments, the archaeon is generally a mesophilic methanogenic archaea. In one alternative of this embodiment, the presence of at least one species from the genera Methanobrevibacter or Methanosphaera is decreased. In another alternative embodiment, the presence of Methanobrevibacter smithii is decreased. In still another embodiment, the presence of Methanosphaera stadtmanae is decreased. In yet another embodiment, the presence of a combination of archaeon genera or species is decreased. By way of non-limiting example, the presence of Methanobrevibacter smithii and Methanosphaera stadtmanae is decreased.

To decrease the presence of any of the archaeon detailed above, methods generally known in the art may be utilized. In one embodiment, a compound having anti-microbial activities against the archaeon is administered to the subject. Non-limiting examples of suitable anti-microbial compounds include metronidzaole, clindamycin, tinidazole, macrolides, and fluoroquinolones. In another embodiment, a compound that inhibits methanogenesis by the archaeon is administered to the subject. Non-limiting examples include 2-bromoethanesulfonate (inhibitor of methyl-coenzyme M reductase), N-alkyl derivatives of para-aminobenzoic acid (inhibitor of tetrahydromethanopterin biosynthesis), ionophore monensin, nitroethane, lumazine, propynoic acid and ethyl 2-butynoate. In yet another embodiment, a hydroxymethylglutaryl-CoA reductase inhibitor is administered to the subject. Non-limiting examples of suitable hydroxymethylglutaryl-CoA reductase inhibitors include lovastatin, atorvastatin, fluvastatin, pravastatin, simvastatin, and rosuvastatin. Alternatively, the diet of the subject may be formulated by changing the composition of glycans (e.g., polyfructose-containing oligosaccharides) in the diet that are preferred by polysaccharide degrading bacterial components of the microbiota (e.g., Bacteroides spp) when in the presence of mesophilic methanogenic archaeal species such as Methanobrevibacter smithii.

Generally speaking, when the archaeon population in the subject's gastrointestinal tract is decreased in accordance with the methods described above, the polysaccharide degrading properties of the subject's gastrointestinal microbiota is altered such that microbial-mediated carbohydrate metabolism or its efficiency is decreased. Typically, depending upon the embodiment, the transcriptome and the metabolome of the gastrointestinal microbiota is altered, as described in the examples. In one embodiment, the microbe is a saccharolytic bacterium. In one alternative of this embodiment, the saccharolytic bacterium is a Bacteroides species. In a further alternative embodiment, the bacterium is Bacteroides thetaiotaomicron. Typically, the carbohydrate will be a plant polysaccharide or dietary fiber. Plant polysaccharides include starch, fructan, cellulose, hemicellulose, and pectin.

Yet another aspect of the invention provides a method for increasing microbial-mediated carbohydrate metabolism or for increasing the efficiency of microbial-mediated carbohydrate metabolism in a subject by altering the archaeon population in the subject's gastrointestinal tract. Because carbohydrate metabolism or the efficiency of carbohydrate metabolism may be increased, the invention also provides methods for promoting weight gain in the subject. Increasing carbohydrate metabolism or the efficiency of carbohydrate metabolism provides methods for treating the symptoms associated with irritable bowel syndrome, which is characterized by the inability to ferment dietary polysaccharides. Changes in the archaeon population may increase microbial-mediated carbohydrate metabolism, whereby increased microbial-mediated carbohydrate metabolism promotes relief of symptoms associated with irritable bowel syndrome.

In accordance with this embodiment, the subject's gastrointestinal archaeon population is altered so as to promote relief of symptoms associated with irritable bowel syndrome in the subject. Typically, the presence of at least one genera of archaeon that resides in the gastrointestinal tract of the subject is increased. In most embodiments, the archaeon is generally a mesophilic methanogenic archaea. In one alternative of this embodiment, the presence of at least one species from the genera Methanobrevibacter or Methanosphaera is increased. In another alternative embodiment, the presence of Methanobrevibacter smithii is increased. In still another embodiment, the presence of Methanosphaera stadtmanae is increased. In yet another embodiment, the presence of a combination of archaeon genera or species is increased. By way of non-limiting example, the presence of Methanobrevibacter smithii and Methanosphaera stadtmanae is increased.

To increase the presence of any of the archaeon detailed above, methods generally known in the art may be utilized. In one embodiment, a suitable probiotic is administered to the subject. Generally speaking, suitable probiotics include those that increase the representation or biological properties of mesophilic methanogenic archaeon that reside in the gastrointestinal tract of the subject. By way of non-limiting example, a probiotic comprising Methanobrevibacter smithii or Methanosphaera stadtmanae, or combinations thereof may be administered to the subject.

Typically, when the archaeon population in the subject's gastrointestinal tract is increased in accordance with the methods described above, the polysaccharide degrading properties of the subject's gastrointestinal microbiota is altered such that microbial-mediated carbohydrate metabolism or its efficiency is increased. In particular, the applicants have discovered that the archaea improve the metabolism of otherwise indigestible dietary polysaccharides by altering the transcriptome and the metabolome of the subject's gastrointestinal microbiota. In one embodiment, the microbe is a saccharolytic bacterium. In one alternative of this embodiment, the saccharolytic bacterium is a Bacteroides species. In a further alternative embodiment, the bacterium is Bacteroides thetaiotaomicron. Typically, the carbohydrate will be a plant polysaccharide or dietary fiber. Plant polysaccharides include starch, fructan, cellulose, hemicellulose, and pectin.

The compounds utilized in this invention to alter the archaeon population may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

The actual effective amounts of compound described herein can and will vary according to the specific composition being utilized, the mode of administration and the age, weight and condition of the subject. Dosages for a particular individual subject can be determined by one of ordinary skill in the art using conventional considerations. Those skilled in the art will appreciate that dosages may also be determined with guidance from Goodman & Gilman's The Pharmacological Basis of Therapeutics, Ninth Edition (1996), Appendix II, pp. 1707-1711 and from Goodman & Gilman's The Pharmacological Basis of Therapeutics, Tenth Edition (2001), Appendix II, pp. 475-493.

### (B) Methods for Treating Weight-Related Disorders

A further aspect of the invention encompasses the use of the methods to regulate weight loss in a subject as a means to treat weight-related disorders. In one embodiment, weight-related disorders are treated by altering the archaeon population in the subject's gastrointestinal tract such that microbial-mediated carbohydrate metabolism in the subject is decreased, as described in (A) above. Decreasing microbial-mediated carbohydrate metabolism, as detailed in this method, promotes weight loss in the subject.

In one particularly preferred embodiment, the weight-related disorder is obesity or an obesity-related disorder. A subject in need of treatment for obesity is diagnosed and is then administered any of the treatments detailed herein, such as in section (A). Typically, a subject in need of treatment for obesity will have at least one of three criteria: (i) BMI over 30; (ii) 100 pounds overweight; or (iii) 100% above an “ideal” body weight. In addition, obesity-related disorders that may be treated by the methods of the invention include metabolic syndrome, type II diabetes, hypertension, cardiovascular disease, and nonalcoholic fatty liver disease.

Another aspect of the invention encompasses a combination therapy to promote weight loss in a subject. In one embodiment, in addition to decreasing the subject's gastrointestinal archaeon population, a composition that promotes weight loss is also administered to the subject. Selection of the appropriate agents for use in combination therapy may be made by one of ordinary skill in the art, according to conventional pharmaceutical principles. Generally speaking, agents will include those that decrease body fat or promote weight loss by a mechanism other the mechanisms detailed herein. In one embodiment, a composition comprising a fasting-induced adipocyte factor (Fiaf) polypeptide may also be administered to the subject. In another embodiment, acarbose may be administered to the subject. Acarbose is an inhibitor of β-glucosidases and is required to break down carbohydrates into simple sugars within the gastrointestinal tract of the subject. In another embodiment, an appetite suppressant such as an amphetamine or a selective serotonin reuptake inhibitor such as sibutramine may be administered to the subject. In still another embodiment, a lipase inhibitor such as orlistat or an inhibitor of lipid absorption such as Xenical may be administered to the subject. The combination of therapeutic agents may act synergistically to decrease body fat or promote weight loss. Using this approach, one may be able to achieve therapeutic efficacy with lower dosages of each agent, thus reducing the potential for adverse side effects.

An additional embodiment of the invention relates to the administration of a composition that generally comprises an active ingredient formulated with a pharmaceutically acceptable excipient. Excipients may include, for example, sugars, starches, celluloses, gums, and proteins. Various formulations are commonly known and are thoroughly discussed in the latest edition of Reminton's Pharmaceutical Sciences (Maack Publishing, Easton Pa.). Such compositions may consist of a Fiaf polypeptide or Fiaf peptidomimetic.

### (C) Biomarkers

A further aspect of the invention provides biomarkers that may be utilized in predicting whether a subject is at risk for becoming obese or suffering from an obesity-related condition. In one embodiment, the biomarker comprises the amount of archaeon in the subject's gastrointestinal tract. In a further embodiment, the biomarker is the representation of archaeon species present in the gastrointestinal tract of the subject. In one embodiment, the archaeon is from the genera Methanobrevibacter or Methanosphaera. In another embodiment, the archaeon is Methanobrevibacter smithii or Methanosphaera stadtmanae.

### DEFINITIONS

The term “altering” as used in the phrase “altering the archaeon population” is to be construed in its broadest interpretation to mean a change in the representation of archaea in the gastrointestinal tract of a subject relative to wild type. The change may be a decrease or an increase in the presence of a particular archaea species.

“BMI” as used herein is defined as a human subject's weight (in kilograms) divided by height (in meters) squared.

GF stands for germ free.

“Metabolome” as used herein is defined as the network of enzymes and their substrates and products, which operate within host or microbial cells under various physiological conditions.

“Subject” as used herein typically is a mammalian species. Non-limiting examples of subjects that may be treated by the methods of the invention include a human, a dog, a cat, a cow, a horse, a rabbit, a pig, a sheep, a goat, as well as non-mammalian species harboring archaea in their guts.

“Transcriptome” as used herein is defined as the network of genes that are being actively transcribed into mRNA in host or microbial cells under various physiological conditions.

As various changes could be made in the above compounds, products and methods without departing from the scope of the invention, it is intended that all matter contained in the above description and in the examples given below, shall be interpreted as illustrative and not in a limiting sense.

### EXAMPLES

It has been difficult to define the mechanisms by which specific members of the microbiota acquire, metabolize and share nutrients with one another and the host. This deficiency reflects the enormous complexity of the intestinal ecosystem. The examples herein utilize a simplified model of the gut ecosystem by raising inbred gnotobiotic mouse strains under germ-free conditions (lacking all microbes) and then colonizing them with one or a defined collection of human-derived microbial symbionts.

### Example 1

### Co-Colonization of Germ-Free Mice with M. Smithii and B. Thetaiotaomicron

To examine the contributions of Archaea to digestive health, age-matched adult germ-free (GF) mice were colonized with the saccharolytic bacterium, Bacteroides thetaiotaomicron alone or in the presence of the methanogen, Methanobrevibacter smithii. Sulfate-reducing bacteria (SRB) serve as alternative consumers of H₂ in the human gut (47, 48). These SRBs are almost exclusively Desulfovibrio spp, with D. piger being the most abundant species in healthy adults (20, 49). D. piger, like M. smithii, is non-saccharolytic; unlike M. smithii, it cannot use formate (50). Therefore, control experiments were performed in which GF mice were colonized with the sulfate-reducing bacterium D. piger alone or in place of M. smithii in the bi-association experiments.

Culture conditions. B. thetaiotaomicron VPI-5482 (ATCC 29148) was cultured anaerobically in TYG (1% tryptone/0.5% yeast extract/0.2% glucose) medium, while M. smithii PS (ATCC 35061) was grown in 125 ml serum bottles (BellCo Glass, Vineland, N.J.) containing 15 mL of Methanobrevibacter complex medium (MBC) supplemented with 3 g/L of formate, 3 g/L of acetate, and 0.3 mL of a freshly prepared, anaerobic solution of filter-sterilized 2.5% Na₂S. The remaining volume in the bottle (headspace) contained a 4:1 mixture of H₂ and CO₂: the headspace was rejuvenated every 1-2 d. M. smithii was also cultured in a BioFlor-110 chemostat with dual fermentation vessels, each containing 750 mL of supplemented MBC, at 37° C. under a constant flow of H₂/CO₂ (4:1). One hour prior to inoculation, 7.5 ml of a sterile 2.5% Na₂S solution was added, followed by half of the contents of a serum bottle culture that had been harvested on day 5 of growth. The chemostat flow rate was maintained at 0.1 L/h (agitation setting, 250 rpm). Sterile 2.5% Na₂S solution (1 mL) was added daily. Aliquots were removed from each vessel at specified times during growth for measurement of OD₆₀₀ and analysis of metabolites. D. piger (ATCC 29098) was cultured anaerobically in Postgate's Medium B.

Colonization of germ-free mice. GF mice belonging to the NMRI/KI inbred strain were housed in gnotobiotic isolators where they were maintained on a strict 12h light cycle (lights on at 0600 h) and fed an autoclaved standard rodent chow diet rich in plant polysaccharides, including polyfructose-containing glycans (fructans) (B&K Universal, East Yorkshire, UK) ad libitum. The mice were colonized with one or more of the following human fecal-derived microbial strains: B. thetaiotaomicron (alone for 14d or 28d); M. smithii (alone for 14d); or B. thetaiotaomicron alone for 14d followed by M. smithii for 14d. The same regimen of mono- and bi-association was followed for B. thetaiotaomicron and D. piger. Each mouse was inoculated with a single gavage with 10⁸ microbes/strain (harvested from overnight stationary phase cultures in the case of B. thetaiotaomicron and D. piger, and from serum bottles after a 5d incubation in the case of M. smithii). Within a given experiment, the same preparation of cultured microbes was used for bi- and mono-association.

Defining the density of colonization. Luminal contents were manually extruded from the cecum and the distal half of the colon immediately after sacrifice, flash frozen in liquid nitrogen, and stored at −80° C. Cells in an aliquot of frozen luminal contents were lysed with bead beating in 2 ml of RLT buffer (Qiagen; 5 min in a Biospec Mini Bead-beater set on maximum). Genomic DNA (gDNA) was then recovered using the QlAgen DNeasy kit and its accompanying protocol. Quantitative PCR was performed using a Mx3000 real-time PCR system (Stratagene). Reaction mixtures (25 μL) contained SYBRGreen Supermix (Bio-Rad), 300 nM of 163 rRNA gene-specific primers (see below), 10 ng of gDNA from cecal contents, or microbial DNA purified from mono-cultures (used as standards). Amplification conditions were 55° C. for 2 min and 95° C. for 15 min, followed by 40 cycles of 95° C. (15 s), 55° C. (45 s), 72° C. (30 s), and 86° C. (20 s). Primer pairs targeted 16S rRNA genes from: B. thetaiotaomicron (Bt. 1F. 5′-ATAGCCTTTCGAAAGRAAGAT-3′ [SEQ ID NO:1]; Bt. 1R, 5′-CCAGTATCAACTGCAATTTTA-3′ [SEQ ID NO:2]; 500 bp product); M. smithii (Msm.1F, 5′-TGAGATGTCCGGCGTTGAA-3′ [SEQ ID NO:3]; Msm.1R, 5′-AAGCCATGCAAGTCGAACGA-3′ [SEQ ID NO:4]; 458 bp product); or D. piger (Dp.1F, 5′-CTAGGGTGTTCTAATCATCATCCTAC-3′ [SEQ ID NO:5]; Dp.1R, 5′-TTGAGTTTCAGCCTTGCGACC-3′ [SEQ ID NO:6]; 481 bp product).

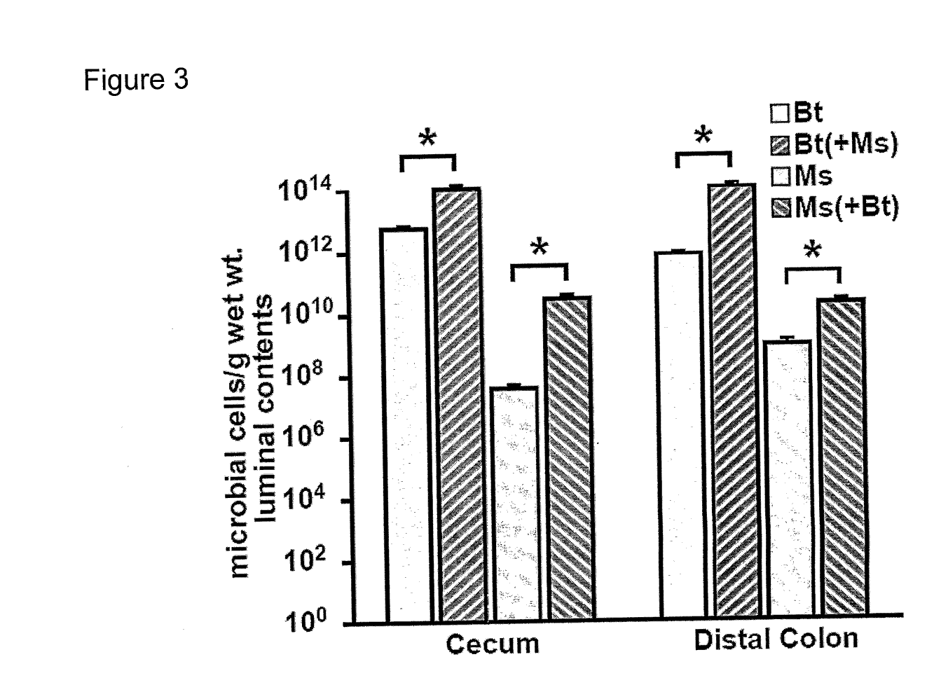

Results. GF mice were reliably and efficiently colonized after a single gavage of 10⁸ M. smithii or B. thetaiotaomicron (mean values: 10¹² organisms/g of cecal contents for B. thetaiotaomicron; 10⁷ for M. smithii; FIG. 3). There were no significant differences in cecal B. thetaiotaomicron levels after 14d or 28d mono-associations (data not shown). Co-colonization (bi-association) with M. smithii and B. thetaiotaomicron resulted in statistically significant (p<0.03) 100 to 1,000-fold enhancement in the density of cecal colonization by both organisms (FIG. 3). The levels of colonization achieved by M. smithii in the ceca and colons of these bi-associated mice were equivalent to those previously reported in the feces of healthy adult humans. In contrast, bi-association of mice with B. thetaiotaomicron and D. piger did not significantly alter cecal or colonic levels of either organism (data not shown). These results suggest that a mutually beneficial relationship is forged between M. smithii and B. thetaiotaomicron in the distal mouse gut that allows them to markedly increase their population size.

### Example 2

### M. Smithii Alters the Dietary Polysaccharide Degradation Pattern of B. Thetaiotaomicron

A combination of whole genome transcriptional profiling and mass spectrometry and microanalytic biochemical assays were utilized to determine the impact of M. smithii on B. thetaiotaomicron nutrient metabolism in vivo, and in particular to determine whether M. smithii modulates the expression of bacterial genes involved in glycan metabolism.

RNA isolation and GeneChip analysis. 100-300 mg of frozen cecal contents (as described above) from each gnotobiotic mouse was added to 2 mL tubes containing 250 μL of 212-300 μm-diameter acid-washed glass beads (Sigma), 500 μL of Buffer A (200 mM NaCl, 20 mM EDTA), 210 μL of 20% SDS, and 500 μL of a mixture of phenol:chloroform:isoamyl alcohol (125:24:1; pH 4.5; Ambion). Samples were lysed using a bead beater (BioSpec; ‘high’ setting for 5 min at room temperature). Cellular debris was pelleted by centrifugation (10,000×g at 4° C. for 3 min). The extraction was repeated by adding another 500 μL of phenol:chloroform:isoamyl alcohol to the aqueous supernatant. RNA was precipitated, resuspended in 100 μL of nuclease-free water (Ambion). After addition of 350 μL Buffer RLT (QlAgen), RNA was further purified using a QlAgen RNeasy mini kit.

cDNA targets for GeneChip hybridization were prepared (www.affymetrix.com/technology/index.affx) from cecal microbial RNA samples isolated from each mouse in each treatment group, and then hybridized to individual custom Affymetrix B. thetaiotaomicron GeneChips containing probesets representing 4,719 of B. thetaiotaomicron's 4,779 predicted protein-coding ORFs (51). These probesets encompass all components of B. thetaiotaomicron's very prominent ‘glycobiome’ (genes involved in carbohydrate acquisition/metabolism/biosynthesis), including 226 predicted glycoside hydrolases, 15 polysaccharide lyases, and 163 paralogs of two outer membrane proteins that bind and import starch (SusC, a malto-oligosaccharide porin, and SusD, which binds starch) (34). All GeneChip datasets were analyzed using DNA-Chip Analyzer v1.3 (dChip; www.biostat.harvard.edu/complab/dchip/). Normalized and modeled (PM-MM) datasets were generated and used to identify differentially expressed genes between the experimental (E) and baseline (B) groups based on the following criteria: E-B>50, E=B p<0.05; ≧33% “Present” call in B; ≧66% “Present” call in E; false discovery rate <3%.

Quantitative RT-PCR analyses were performed using methods similar to the qPCR assay described above, with the exception that each reaction contained 10 ng of cDNA template and uracil-DNA glycosidase (0.01 U/μL). All amplicons were 100-120 bp in length.

Analysis of cecal glycans. Gas chromatography-mass spectrometry (GC-MS) analyses were used to determine the levels of neutral and amino sugars in cecal glycans (51). Fructan levels were assayed using a different microanalytic approach (52). Cecal samples were collected with a 10 μL inoculation loop, freeze dried at −35° C. for 4 d, weighed, and stored under vacuum at −80° C. until use (stable for at least one month). Samples (10-15 mg) were then homogenized at 1° C. in 0.25 mL of 1% oxalic acid (prepared in H₂O) and divided into two equal-sized aliquots, one of which was heated to 100° C. for 30 min (acid hydrolysis sample), while the other was maintained at 1° C. (control sample). A 10 μL aliquot of each sample was added to a 1 mL solution containing 50 mM Tris HCl pH 8.1, 1 mM MgCl₂, 0.02% BSA, 0.5 mM ATP, 0.1 mM NADP+, 2 μg/mL Leuconostoc mesenteroides glucose-6 phosphate dehydrogenase (253 units/mg protein; Calbiochem), 10 μg/mL yeast hexokinase (50 units/mg protein; Sigma) and 10 μg/mL yeast phosphoglucose isomerase (500 units/mg protein; Sigma). Glucan levels were measured in a similar manner to fructans except that phosphoglucose isomerase was omitted from the reactions. The mixture was subsequently incubated for 30 min at 24° C. The resulting NADPH product was detected using a fluorimeter. Fructose or glucose standards (5-10 nmol) were carried through all steps.

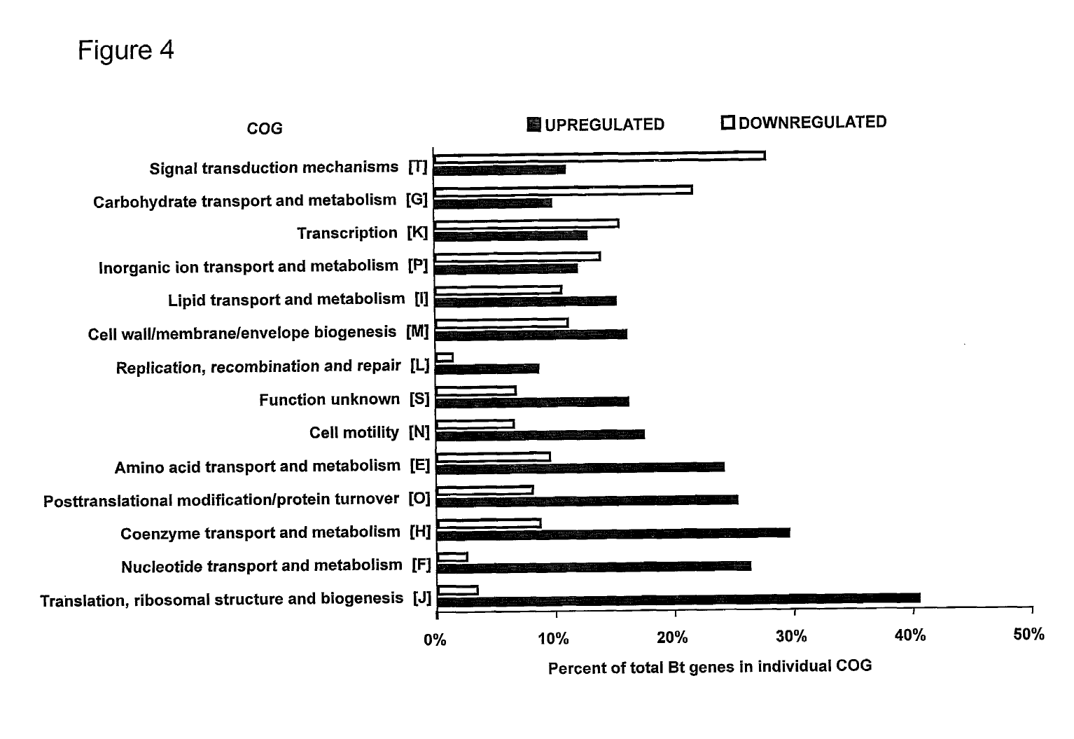

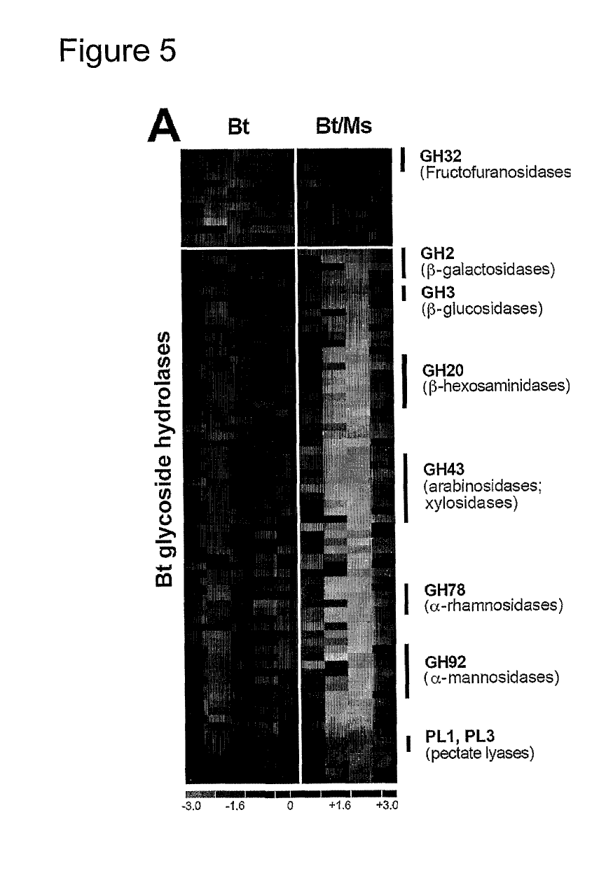

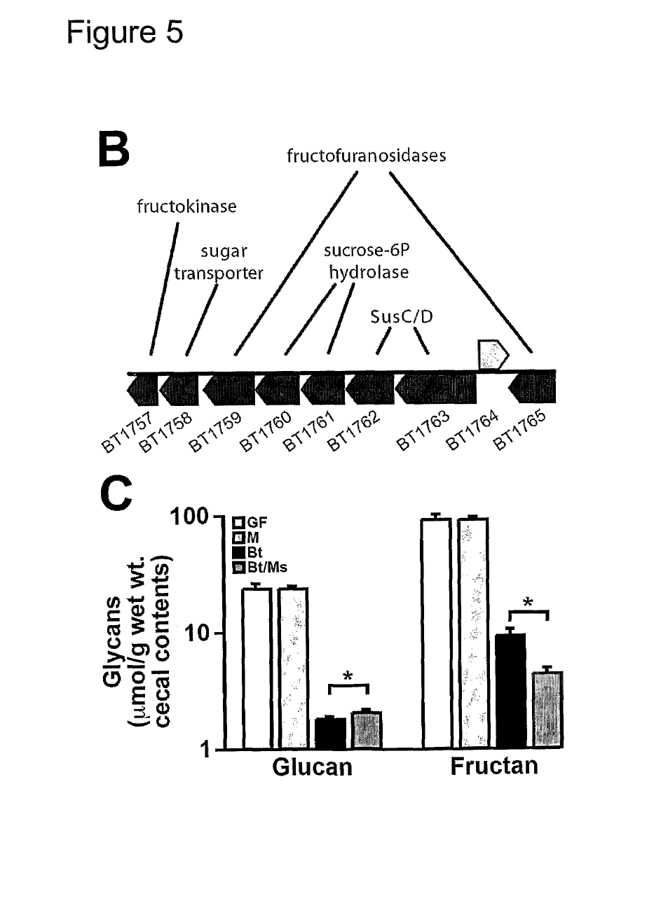

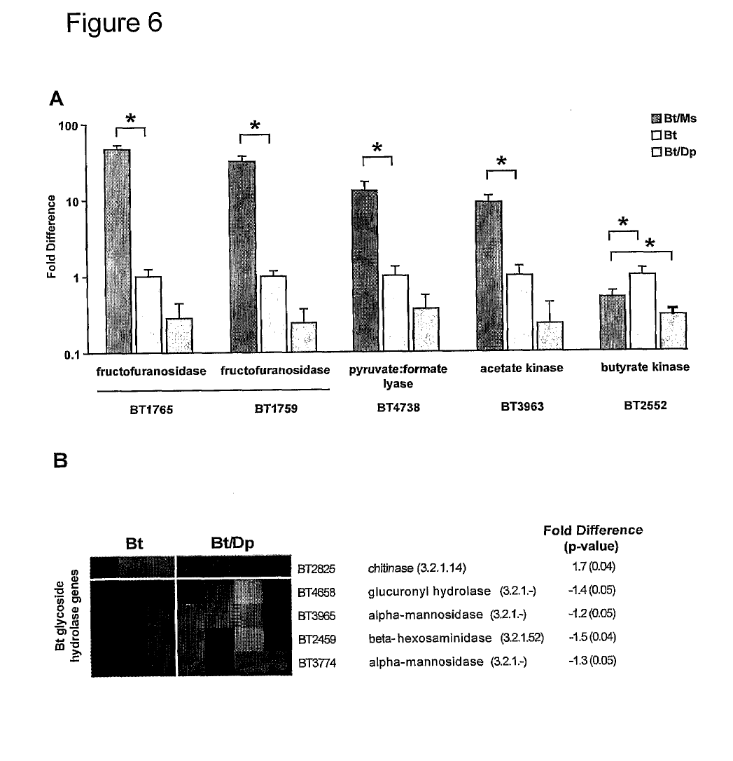

Results. Unsupervised hierarchical clustering of the resulting GeneChip datasets revealed that colonization of the cecal habitat with M. smithii dramatically alters B. thetaiotaomicron's transcriptome: 638 genes were defined as significantly upregulated and 462 genes as significantly downregulated compared with their levels of expression during a 14d B. thetaiotaomicron mono-association (p<0.05). The regulated genes were placed into Clusters of Orthologous Groups (COGs). Co-colonization with M. smithii upregulates B. thetaiotaomicron genes involved DNA replication and protein production, which is consistent with the enhanced representation of B. thetaiotaomicron in the distal gut (FIG. 4). The presence of M. smithii also causes B. thetaiotaomicron to downregulate expression of many genes involved in carbohydrate metabolism (FIG. 4) including 70 glycoside hydrolases (e.g., arabinosidases, xylosidases, glucosidases, galactosidases, mannosidases, rhamnosidases and pectate lyases). There is an accompanying marked induction of three fructofuranosidases (FIG. 5A). Two of these fructan-degrading glycoside hydrolases are encoded by ORFs situated in a gene cluster (BT1757-BT1765) that includes a putative sugar transporter, SusC/SusD paralogs, and the organism's only fructokinase (FIG. 5B). Augmented expression of this cluster was validated by qRT-PCR (FIG. 6A). There were 32±5.8 and 47±5.9-fold increases for the fructofuranosidases (BT1759 and BT1765, respectively) and a 6.4±2.8-fold increase for the fructokinase (BT1757).

Fructose is easily shunted into the glycolytic pathway via fructokinase, making fructans desirable energy sources. This notion is supported by GeneChip analyses of B. thetaiotaomicron grown in chemostats containing glucose and a complex mixture of polysaccharides (TYG medium). Expression of the polyfructose degradation cluster peaked in early log phase with 7.5- to 53.2-fold higher levels for BT1757-BT1765 transcripts compared to late log/stationary phase where B. thetaiotaomicron utilizes less coveted glycans such as mannans (datasets from 51).

In contrast, co-colonization with D. piger did not produce a significant change in expression of these fructofuranosidases, or the fructokinase, as judged by GeneChip and qRT-PCR assays (FIG. 6). Overall, D. piger had very modest effects on the B. thetaiotaomicron transcriptome: of the 41 differentially expressed genes only four were glycoside hydrolases (two α-mannosidases, a β-hexosaminidase and a glucoronyl hydrolase; all were downregulated) (FIG. 6B).

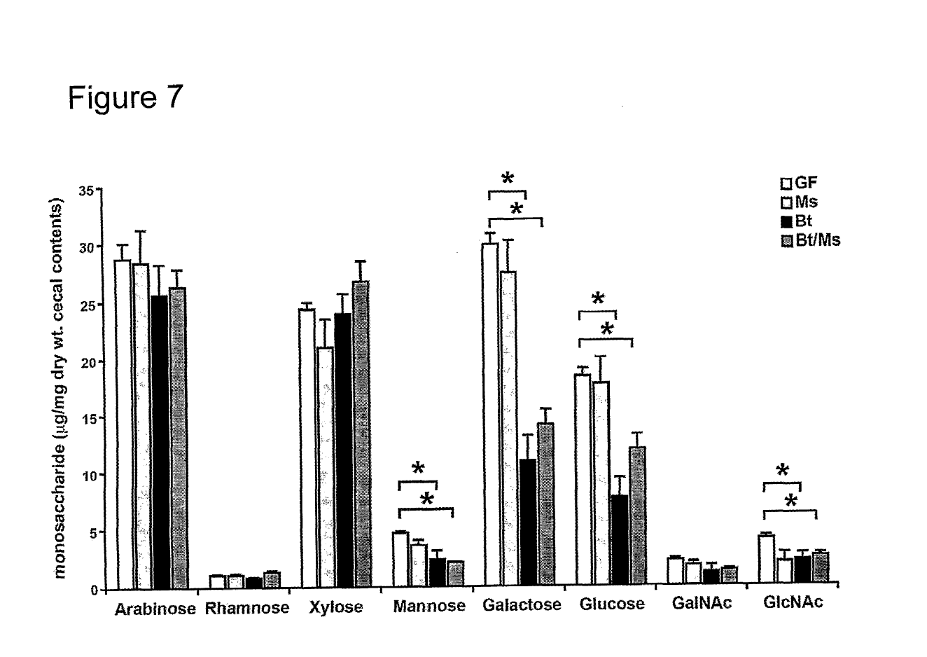

Biochemical studies of cecal contents recovered from GF mice fed a polysaccharide-rich diet revealed that fructans were 3.8-fold higher than polyglucose-containing glycans (glucans) (85±6 vs. 25±2 μmol/g wet weight of contents; p<0.005). Consistent with the in vitro and in vivo transcriptional profiling results, biochemical assays demonstrated a statistically significant 52±4% decrease in cecal fructan levels after B. thetaiotaomicron/M. smithii co-colonization (compared to B. thetaiotaomicron mono-associated mice; p<0.05; FIG. 5C). Glucans increased modestly (15±3%; p<0.05; FIG. 5C), indicating continued albeit slightly reduced digestion of glucose-containing polysaccharides. GC-MS analysis of neutral and amino sugars released by acid hydrolysis of cecal contents, revealed that bi-association produced modest, but not statistically significant, decreases in the consumption of these carbohydrates compared to the B. thetaiotaomicron mono-associated state), suggesting that increased consumption of fructans does not demand forfeiture of the consumption of other polysaccharides (FIG. 7).

### Example 3

### M. Smithii Alters the Metabolome of B. Thetaiotaomicron Toward Increased Production of Acetate and Formate

Whole genome transcriptional profiling (as above) and mass spectrometry assays were employed to determine the impact of M. smithii on B. thetaiotaomicron fermentative metabolism in vivo.

Assays of organic acids. SCFAs in mouse serum (see below) and cecal samples were assayed using a modification of the method of Moreau et al. (53). For analysis of sera, mice were fasted for 4 h, blood was collected by retro-orbital phlebotomy into serum separation tubes (Becton Dickinson), spun, and the supernatant (serum) was stored at −80° C. prior to assay. To assay, 50 μL of serum, or 100-200 mg of frozen cecal contents, were transferred to a 4 mL glass vial fitted with a septum cap PTFE liner (National Scientific), and containing 10 μL of stock solution of internal standards (Isotec; each of the following components at 20 mM: [²H₂]- and [1-¹³C]acetate, [²H₅]propionate, and [¹³C₄]butyrate). Following acidification with 10 μL of 37% HCl, SCFAs were extracted (2 mL diethyl ether/extraction; 2 cycles). The upper organic layer from each extraction was recovered and pooled. For derivatization, a 60 μL aliquot of the extracted sample was mixed together with 20 μL of N-tert-butyldimethylsilyl-N-methyltrifluoracetamide (MTBSTFA; Sigma) at room temperature. An aliquot (2 μL) of the derivatized sample was injected into a gas chromatograph (Hewlett Packard 6890) coupled to a mass spectrometer detector (Agilent 5973). Analyses were completed using DB-5MS (60 m, 0.25 mm i.d., 0.25 um film coating; P. J. Cobert, St. Louis, Mo.) and electronic impact (70 eV) for ionization. A linear temperature gradient was used. The initial temperature of 80° C. was held for 1 min, then increased to 280° C. (15° C./min) and maintained at 280° C. for 5 min. The source temperature and emission current were 200° C. and 300 μA, respectively. The injector and transfer line temperatures were 250° C. Quantitation was completed in selected ion monitoring acquisition mode by comparison to labeled internal standards [formate was also compared to [²H₂]- and [1-¹³C]acetate]. The m/z ratios of monitored ions were: 103 (formic acid), 117 (acetic acid), 131 (propionic acid), 145 (butyric acid), 121([²H₂]- and [1-¹³C]acetate), 136 ([²H₅]propionate) and 149 ([¹³C₄]butyrate).

Organic anions were analyzed in in vitro cultures using a Dionex 600X Ion Chromatograph (IC). The analytes were separated on a Dionex AS11-HC column and detected with a Dionex ED50 Electrochemical Detector using suppressed conductivity with multistep gradient program and 1.5 to 60 mM potassium hydroxide as the eluent. The eluent was generated by a Dionex EG40 Eluent Generator equipped with a Dionex Potassium Hydroxide EluGen cartridge. The IC was calibrated from 0.5 to 10 ppm for all analytes. Detection limits using this method are 0.1 ppm for the six organic anions.

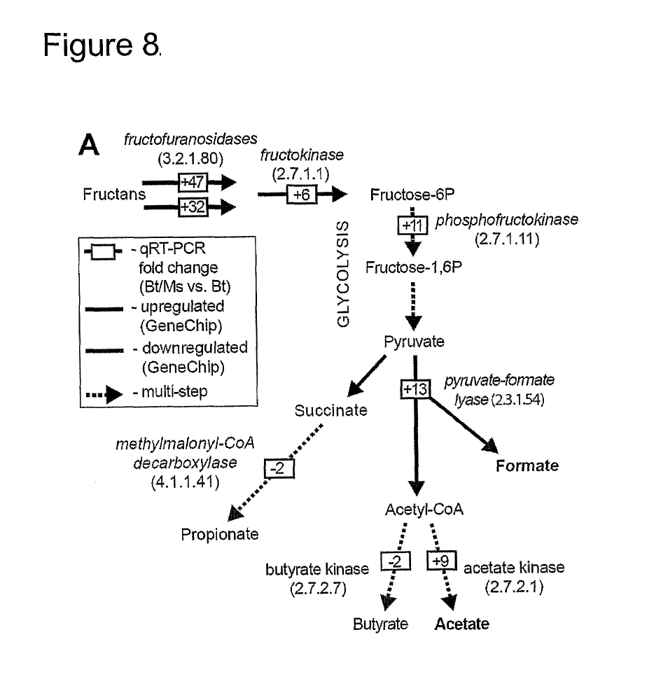

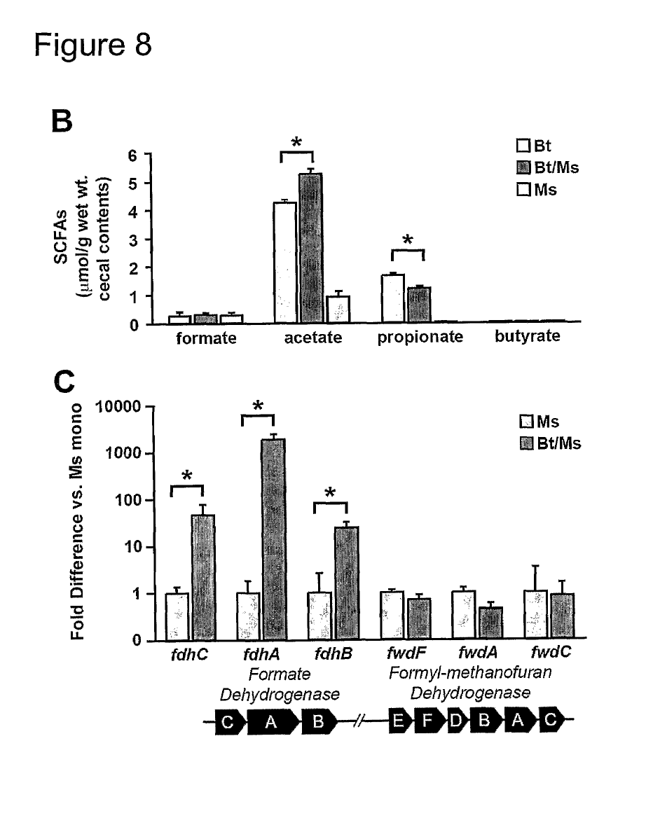

Results. In silico reconstructions of the B. thetaiotaomicron metabolome, obtained by placing the predicted enzyme products of bacterial genes responsive to the presence of M. smithii onto KEGG metabolic maps, indicated that co-colonization produces a shift in gene expression towards increased production of acetate and formate, and reduced production of butyrate and propionate (FIG. 8A). Follow-up GC-MS analysis of cecal SCFA levels confirmed a significant increase in acetate, and a significant decrease in propionate in bi-associated compared to B. thetaiotaomicron mono-associated mice (p<0.02; FIG. 8B). Cecal formate levels, however, were not significantly different between bi- and mono-associated animals (FIG. 8B).

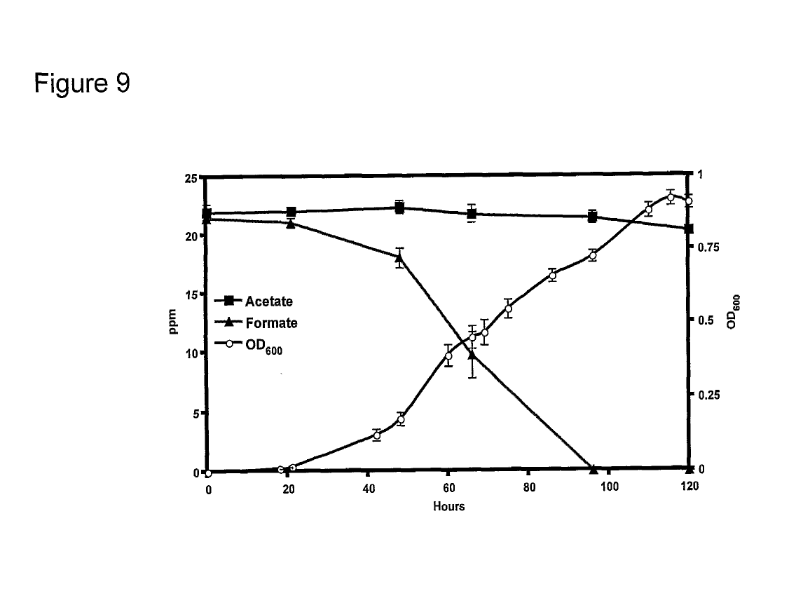

While H₂ is generally viewed as the principal currency for bacterial-archaeal electron transfer, formate can serve an analogous role: (i) it has greater solubility than H₂ in aqueous environments; (ii) there is almost no difference in the energetic couples for CO₂/formate and H+/H₂ [−420 and −414 mV, respectively]; and (iii) ferrodoxin-linked electron transfer components allow inter-conversion of formate and H₂ by methanogenic archaea. It was found that during in vitro growth in acetate and formate-supplemented rich medium, M. smithii preferentially consumed formate (FIG. 9). This raised the possibility that augmented formate production by B. thetaiotaomicron in vivo is masked by its utilization by M. smithii. Evidence for in vivo formate consumption by M. smithii came from additional experiments based on the current draft sequence of its genome, which revealed a gene cluster consisting of a formate transporter (fdhC), formate dehydrogenase subunits (fdhAB), and the subunits of tungsten-containing formylmethanofuran dehydrogenase (fwdEFDBAC; the first enzyme in the methanogenesis pathway) (FIG. 8C). Quantitative RT-PCR established that M. smithii transcripts encoding FdhC, FdhA, and FdhB were expressed at 48±3, 1882±559, and 25±8-fold higher levels, respectively, when B. thetaiotaomicron was present (FIG. 8C). Formylmethanofuran dehydrogenase was constitutively expressed and not affected by bi-association (FIG. 8C).

These findings reveal some of the underpinnings of M. smithii-B. thetaiotaomicron mutualism. B. thetaiotaomicron obtains energy from facilitated fermentation of coveted glycans (fructans) and increased production of acetate (yields more ATP than other end products of fermentation). This allows a larger population of B. thetaiotaomicron to be supported (FIG. 3). M. smithii, in turn, benefits by obtaining formate from B. thetaiotaomicron for methanogenesis, and its population expands (FIG. 3).

### Example 4

### Co-Colonization of Mice with M. Smithii and B. Thetaiotaomicron Enhances Host Energy Storage

Colonic absorption of SCFAs generated during fermentation represents at least 10% of our daily caloric intake (54). To determine how a two-component model microbiota consisting of M. smithii and B. thetaiotaomicron affects host energy balance, serum SCFA levels, liver triglyceride levels, and body fat content were measured.

Methods. SCFA measurements were completed as above. Isolation of liver RNA was completed according to manufacturer's protocols (Qiagen RNeasy). Total body fat content was measured in 12-week old male NMRI mice using dual-energy x-ray absorptiometry (Lunar PIXImus Mouse, GE Medical Systems, Waukesha, Wis.) as previously described (6). Epididymal fat pads and livers were removed and weighed. A portion of the liver was assayed for triglyceride content using a standard biochemical method

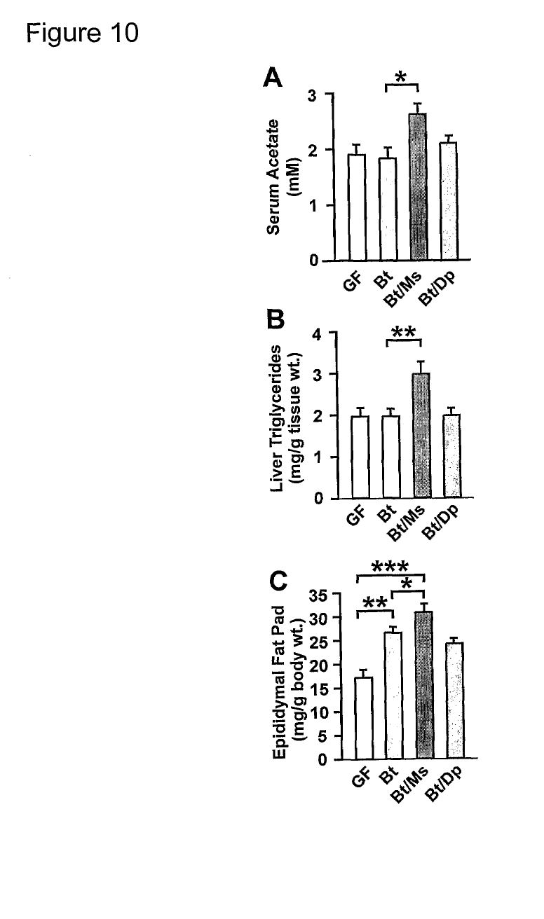

Results. B. thetaiotaomicron/M. smithii bi-associated mice exhibit increased recovery and storage of dietary calories. As in the cecum, addition of M. smithii produced significantly greater serum acetate levels compared with B. thetaiotaomicron mono-associated controls, though no significant increases occurred with addition of D. piger (FIG. 10A). Distal gut-derived SCFAs are transported, via the portal vein, to the liver where they stimulate de novo lipogenesis; a key enzyme in this process is fatty acid synthase (Fas). Quantitative RT-PCR studies revealed that compared to GF animals, Fas gene expression was increased by 142±13% in B. thetaiotaomicron/M. smithii versus 61±9% for B. thetaiotaomicron mono-associated mice (p<0.03). Biochemical assays showed that addition of M. smithii, but not D. piger, to B. thetaiotaomicron-colonized animals produced significant increases in total liver triglyceride levels (FIG. 10B).

The increase in hepatic de novo lipogenesis was accompanied by increased storage of energy in fat cells. Epididymal fat pad weights were significantly greater in B. thetaiotaomicron/M. smithii bi-associated mice compared to B. thetaiotaomicron mono-associated controls [80±6% increase over GF versus 54±7%; p<0.01; FIG. 10C]. In contrast, there was no significant difference in fat pad weights between the B. thetaiotaomicron/D. piger and B. thetaiotaomicron groups (FIG. 10C). Dual-energy x-ray absorptiometry (DEXA) independently confirmed these findings: compared with GF mice, total body fat stores were increased 47±4% in B. thetaiotaomicron/M. smithii bi-associated versus 34±3% in B. thetaiotaomicron mono-associated animals (n=5/group; p<0.05). The increase in adiposity was not accompanied by any statistically significant differences in chow consumption (data not shown). In addition, total body weight did not change significantly (data not shown), a finding explained by the well-documented reduction in cecal weight that occurs after colonization of gnotobiotic animals (55).

The study indicates that the representation of methanogenic archaea in an individual's gut microbiota may affect energy harvest from dietary glycans as well as host energy storage. These experiments demonstrate that M. smithii acts as a ‘power broker’ in the distal gut community, regulating the specificity of polysaccharide fermentation, and influencing the amount of calories deposited in fat stores.

### REFERENCES

All references cited in the preceding text of the patent application or in the following reference list, to the extent that they provide exemplary, procedural, or other details supplementary to those set forth herein, are specifically incorporated by reference to the same extent as if each individual publication or patent application was specifically and individually indicated to be incorporated by reference.
1. C. Bouchard, N Engl J Med 343, 1888-1889 (2000).
2. I. R. Wanless, J. S. Lentz, Hepatology 12, 1106-1110 (1990).
3. J. F. Silverman et al., Am J Gastroenterol 85, 1349-1355 (1990).
4. B. A. Neuschwander-Tetri, S. H. Caldwell, Hepatology 37, 1202-1219 (2003).
5. A. A. Salyers, P. Valentine, V. Hwa, in Genetics and Molecular Biology of Anaerobic Bacteria, E. Sebald, Ed. (Springer-Verlag, New York), pp. 505-516 (1993).
6. F. Backhed et al., Proc Natl Acad Sci USA 101, 15718-15723 (2004).
7. P. B. Hylemon, J. Harder, FEMS Microbiol Rev 22, 475-488 (1998).
8. S. H. Duncan et al., Appl Environ Microbiol 68, 3841-3847 (2002).
9. M. J. Hill, Eur J Cancer Prev 6, S43-S45 (1997).
10. C. Braun-Fahrlander et al., N Engl J Med 347, 869-877 (2002).
11. D. L. Topping, P. M. Clifton, Physiol Rev 81, 1031-1064D (2001).
12. C. R. Woese, O. Kandler, M. L. Wheelis, Proc Natl Acad Sci USA 87, 4576-4579 (1990).
13. B. Morvan, F. Bonnemoy, G. Fonty, P. Gouet, Curr Microbiol 32, 129-133 (1996).
14. C. Lin, T. L. Miller, Arch Microbiol 169, 397-403 (1998).
15. A. Brauman et al., FEMS Microbiol Ecol 35, 27-36 (2001).
16. M. F. Whitford, R. M. Teather, R. J. Forster, BMC Microbiol 1, 5 (2001).
17. I. Segal, A. R. Walker, S. Lord, J. H. Cummings, Gut 29, 608-613 (1988).
18. M. J. Hudson, A. M. Tomkins, H. S. Wiggins, B. S. Drasar, Scand J Gastroenterol 28, 993-998 (1993).
19. J. H. Hackstein, T. A. Van Alen, H. Op Den Camp, A. Smits, E. Mariman, Dtsch Tierarztl Wochenschr 102, 152-154 (1995).
20. P. B. Eckburg et al., Science 308, 1635-1638 (2005).
21. P. Pochart et al, Gastroenterology 105, 1281-1285 (1993).
22. L. El Oufir et al., Gut 38, 870-877 (1996).
23. T. L. Miller, M. J. Wolin, Syst Appl Microbiol 7, 223-229 (1986).
24. T. L. Miller, M. J. Wolin, Arch Microbiol 131, 14-18 (1982).
25. F. Rieu-Lesme, C. Delbes, L. Sollelis, Curr Microbiol 51, 317-321 (2005).
26. M. Blaut, Antonie Van Leeuwenhoek 66, 187-208 (1994).
27. J. N. Reeve, Annu Rev Microbiol 46, 165-191 (1992).
28. U. Deppenmeier et al., J Mol Microbiol Biotechnol 4, 453-461 (2002).
29. J. E. Galagan et al., Genome Res 12, 532-542 (2002).
30. T. L. Miller, M. J. Wolin, E. C. de Macario, A. J. Macario, Appl Environ Microbiol 43, 227-232 (1982).
31. L. V. Hooper, T. Midtvedt, J. I. Gordon, Annu Rev Nutr 22, 283-307 (2002).
32. A. A. Salyers, G. Bonheyo, N. B. Shoemaker, Methods 20, 35-46 (2000).
33. W. E. Moore, L. V. Holdman, Appl Microbiol 27, 961-979 (1974).
34. J. Xu et al., Science 299, 2074-2076 (2003).
35. J. L. Sonnenburg et al., Science 307, 1955-1959 (2005).
36. T. L. Miller, Arch Microbiol 117, 145-152 (1978).
37. J. M. Macy, L. G. Ljungdahl, G. Gottschalk, J Bacteriol 134, 84-91 (1978).
38. N. Pan, J. A. Imlay, Mol Microbiol 39, 1562-1571 (2001).
39. B. Schink, Microbiol Mol Biol Rev 61, 262-280 (1997).
40. A. J. Stams, Antonie Van Leeuwenhoek 66, 271-294 (1994).
41. S. U. Christl, P. R. Murgatroyd, G. R. Gibson, J. H. Cummings, Gastroenterology 102, 1269-1277 (1992).
42. B. Schink, Antonie Van Leeuwenhoek 81, 257-261 (2002).
43. J. L. Rychlik, T. May, Curr Microbiol 40, 176-180 (2000).
44. T. L. Miller, E. Currenti, M. J. Wolin, Appl Microbiol Biotechnol 54, 494-498 (2000).
45. C. Robert, C. Del'Homme, A. Bernalier-Donadille, FEMS Microbiol Lett 205, 209-214 (2001).
46. C. S. Stewart, A. J. Richardson, R. M. Douglas, C. J. Rumney, in Microbiology and Biochemistry of Strict Anaerobes Involved in Interspecies Transfer J.-L. Garcia, Ed. (Plenum Press, New York) pp. 121-131 (1990).
47. A. Strocchi, J. Furne, C. Ellis, M. D. Levitt, Gut 35, 1098-1101 (1994).
48. S. U. Christl, P. R. Murgatroyd, G. R. Gibson, J. H. Cummings, Gastroenterology 102, 1269-1277 (1992).
49. J. Loubinoux et al., Int J Syst Evol Microbiol 52, 1305-1308 (2002).
50. W. E. Moore, J. L. Johnson, L. V. Holdeman, Int J Syst Bacteriol 26, 238-252 (1976).
51. J. L. Sonnenburg et al., Science 307, 1955-1959 (2005).
52. J. V. Passonneau, O. H. Lowry, Enzymatic Analysis: A Practical Guide (Humana Press, Totawa, N.J.) (1993).
53. N. M. Moreau et al., J Chromatogr B Analyt Technol Biomed Life Sci 784, 395-403 (2003).
54. N. I. McNeil, Am J Clin Nutr 39, 338-342 (1984).
55. B. Wostmann, E. Bruckner-Kardoss, Am J Physiol 197, 1345-1346 (1959).
56. W. F. Fricke et al., J Bacteriol 188(2), 642-658 (2006).

1. A method for promoting weight loss in a subject, the method comprising altering the archaeon population in the subject's gastrointestinal tract such that microbial-mediated carbohydrate metabolism is decreased in the subject.

2. The method of claim 1, wherein carbohydrate metabolism is mediated by a saccharolytic bacterium.

3. The method of claim 1, wherein carbohydrate metabolism is mediated by a Bacteroides species.

4. (canceled)

5. The method of claim 1, wherein the archaeon population is altered by decreasing the presence of at least one genera that resides in the gastrointestinal tract of the subject.

6. The method of claim 1, wherein the archaeon population is altered by decreasing the presence of at least one species from the genera Methanobrevibacter or Methanosphaera.

7. (canceled)

8. The method of claim 5, wherein the presence of an archaeon genera is decreased by administering a compound selected from the group consisting of compounds having anti-microbial activities against the archaeon, compounds having anti-methanogenic activities against the archaeon, or a hydroxymethylglutaryl-CoA reductase inhibitor.

9-11. (canceled)

12. A method for modulating carbohydrate metabolism in a subject, the method comprising altering the archaeon population in the subject's gastrointestinal tract such that microbial-mediated carbohydrate metabolism is modulated in the subject.

13. The method of claim 12, wherein carbohydrate metabolism is mediated by a saccharolytic bacterium.

14. The method of claim 12, wherein carbohydrate metabolism is mediated by a Bacteroides species.

15. (canceled)

16. The method of claim 12, wherein the archaeon population is altered by decreasing the presence of at least one genera that resides in the gastrointestinal tract of the subject.

17. The method of claim 12, wherein the archaeon population is altered by decreasing the presence of at least one species from the genera Methanobrevibacter or Methanosphaera.

18. (canceled)

19. The method of claim 16, wherein the presence of an archaeon genera is decreased by administering a compound selected from the group consisting of compounds having anti-microbial activities against the archaeon, compounds having anti-methanogenic activities against the archaeon, or a hydroxymethylglutaryl-CoA reductase inhibitor.

20-39. (canceled)

40. A method for treating obesity or an obesity-related disorder, the method comprising: (a) diagnosing a subject in need of treatment for obesity or an obesity-related disorder; and (b) altering the archaeon population in the subject's gastrointestinal tract such that microbial-mediated carbohydrate metabolism is decreased in the subject.

41. The method of claim 40, wherein carbohydrate metabolism is mediated by a saccharolytic bacterium.

42. The method of claim 40, wherein carbohydrate metabolism is mediated by a Bacteroides species.

43. (canceled)

44. The method of claim 40, wherein the archaeon population is altered by decreasing the presence of at least one genera that resides in the gastrointestinal tract of the subject.

45. The method of claim 40, wherein the archaeon population is altered by decreasing the presence of at least one species from the genera Methanobrevibacter or Methanosphaera.

46. (canceled)

47. The method of claim 44, wherein the presence of an archaeon genera is decreased by administering a compound selected from the group consisting of compounds having anti-microbial activities against the archaeon, compounds having anti-methanogenic activities against the archaeon, or a hydroxymethylglutaryl-CoA reductase inhibitor.

48-63. (canceled)

64. The method of claim 1, wherein the decreased microbial-mediated carbohydrate metabolism decreases storage of energy in fat cells in the subject.

65. The method of claim 40, wherein the decreased microbial-mediated carbohydrate metabolism decreases storage of energy in fat cells in the subject.
